(19) United States
(12) Patent Application Publication
(10) Pub. No.: US 2012/0004597 A1
(43) Pub. Date: Jan. 5, 2012

(54) MEDICAL DEVICE FOR INJECTING FLUID INTO A BODY

(75) Inventor: Eric PEROUSE, Paris (FR)

(73) Assignee: PEROUSE MEDICAL, IVRY LE TEMPLE (FR)

(21) Appl. No.: 13/176,212

(22) Filed: Jul. 5, 2011

(30) Foreign Application Priority Data
Jul. 5, 2010 (FR) 10 55428

(51) Int. Cl. A61M 37/00; A61M 5/32

(52) U.S. Cl. 604/22; 604/198; 604/26

(57) ABSTRACT

The medical device (10) has a prop (12) extending between a proximal end (12A) and a distal end, a nose (16) in the form of a perforating tip, secured to the distal end of the prop (12), a sheath (18), in which the prop (12) is housed, which can be moved by sliding between a first position in contact with the nose (16), and a second position spaced away from the nose (16), and at least one tube (22) extending in the sheath (18) between a proximal end (22A), accessible outside the sheath (18), and a distal end (22B) secured to the nose (16), the tube (22) having at least one perforation (24) near its distal end (22B). Each tube (22) can be radially deployed between a retracted configuration, in which each tube (22) can be covered by the sheath (18), and a radially protruding deployed configuration.

The present invention relates to a medical device for injecting a fluid, in particular a medical substance, into a body, and in particular into a tumor.

In cancer treatment, cancerous tumors present in the body are sometimes surgically removed by making an incision in the patient. Alternatively, it is known to inject a fluid into the body, near the tumor, so as to reduce the tumor.

To that end, already known in the state of the art is a medical injection device, comprising a conduit making it possible to inject vapor, or any other suitable medical substance, near a tumor, in particular to destroy the tumor. Such a device is in particular used to treat liver cancer.

The effectiveness of such a device is generally limited, in that it does not allow easy injection of the medical substance in a sufficiently widespread manner in the tumor.

The present invention in particular aims to resolve this drawback, by providing a medical injection device that is easy to use and whereof the effectiveness is improved relative to a traditional device.

To that end, the invention in particular relates to a medical device, in particular to inject a fluid into a body, characterized in that it includes:
a prop extending between a proximal end and a distal end,
a nose in the form of a perforating tip, secured to the distal end of the prop,
at least one injection tube for injecting fluid into the body, the tube extending between a proximal end and a distal end, the tube having at least one perforation near its distal end, and
a sheath, in which the prop and the tube are housed, the sheath being able to be moved by sliding along the prop, between a first position in which the sheath covers the perforation, and a second position in which the perforation is exposed,
and in which:
when the sheath is in its second position, each tube can be deployed radially between a retracted configuration, in which each tube can be covered by the sheath, and a radially protruding deployed configuration, and
the proximal end of each tube can be accessed outside the sheath, so that the deployment of each tube towards its deployed position can be controlled from its proximal end, the distal end of each tube being secured to the nose or the prop.

When the device is installed, percutaneously, into a patient's body so that the nose has pierced a tumor, and the sheath is moved into its second position, the distal end of the tube is installed in the tumor.

By manipulating the tube from its proximal end, while keeping the nose and the sheath immobile, the tube can be deployed radially in the tumor, then extending into the tumor.

When the medical substance is injected by the perforations of the tube, this substance is then injected in a relatively widespread manner in the tumor. It will be noted that the extent of the injection is still further increased if the device has a plurality of tubes, which then deploy in different directions.

Optionally, a medical device according to the invention has one or more of the following features, considered alone or according to all technically possible combinations:
the device has at least two fluid injection tubes, and means for jointly moving each tube by sliding in the sheath, accessible at the proximal end of the tubes,
the device has a means for immobilizing the sheath in its second position relative to the prop,
the sheath is close to the nose in its first position, preferably in contact with said nose, and the sheath is spaced away from this nose in the second position,
each tube has, near its distal end, a cutting means, for example having a blade, able to cut out a tumor when said tube deploys radially in the tumor,
at least one element from amongst the prop, the tube or the sheath is provided with graduations, indicating the movement of the tube or sheath relative to the prop,
the nose is made from a radiopaque material, and
the device has a fluid injection means intended to be connected to the proximal end of each tube, for example comprising a vapor generator connected to a pump or a syringe.

The invention also relates to a method of using the medical device as previously defined, having the following steps:
inserting the medical device into the patient, until the nose reaches a tumor to be treated,
manipulating the nose such that said nose pierces the tumor and passes all the way through it,
moving the sheath towards its second position,
radially deploying the tube in the tumor,
injecting fluid through the perforation.

The invention will be better understood upon reading the following description, provided solely as an example and done in reference to the appended figures, in which:

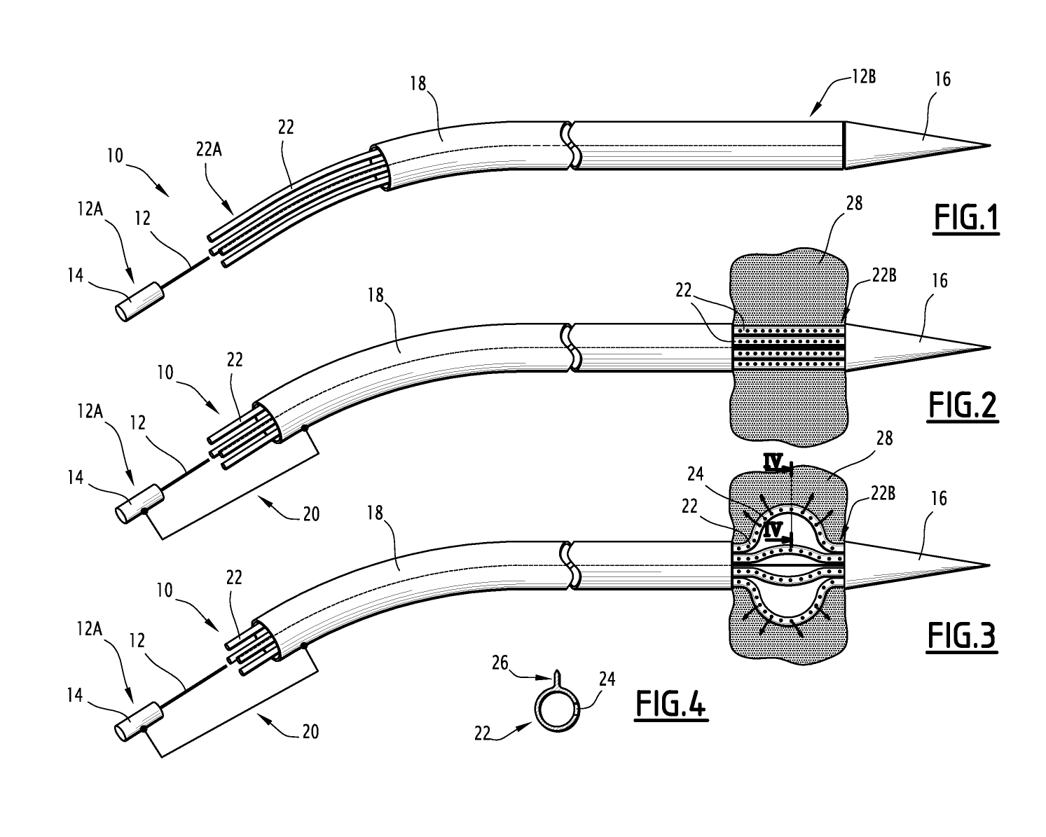

FIGS. 1 to 3 diagrammatically show a medical device according to one embodiment of the invention, during three respective steps for implementing this device;

FIG. 4 is a diagrammatic transverse cross-sectional view, along arrows IV-IV, of the medical device of FIG. 3.

FIGS. 1 to 3 show a medical device 10, in particular for injecting a medical substance into a tumor. The medical substance for example includes vapor.

The medical device 10 has a prop 12, extending between a proximal end 12A and a distal end 12B.

This prop 12 is secured, at its proximal end 12A, with a handling member 14, and at its distal end 12B, with a nose 16 in the form of a perforating tip.

The nose 16 is in particular intended to pierce a tumor, as will be described later. Preferably, the nose 16 is made from a radiopaque material, so that the movement of this nose 16 in a patient's body can be observed by radiography.

The medical device 10 also has a sheath 18, in which the prop 12 is housed. The sheath 18 can be moved by sliding along the prop 12, between a first position in which the sheath 18 is close to the nose 16, for example in contact with the nose 16 (shown in FIG. 1) and a second position in which the sheath is spaced away from this nose 16 (shown in FIGS. 2 and 3).

Advantageously, the medical device 10 has a means 20 for immobilizing the sheath 18 in its second position relative to the prop 12.

The medical device 10 also has at least one tube 22, preferably a plurality of tubes 22, extending partially in the sheath 18 between a proximal end 22A and a distal end 22B that is secured to the nose 16. Each tube 22 is advantageously more flexible than the sheath 18.

Each tube 22 has, near its distal end 22B, at least one perforation 24, preferably a plurality of perforations 24 spaced along the tube 22, through which the medical substance is intended to be injected.

The proximal end 22A of each tube 22 is accessible outside the sheath 18, even when this sheath 18 is in its second position. In this way it is possible to manipulate the tubes 22 from their proximal end 22A.

To that end, the medical device 10 has a means for moving each tube 22 by sliding in the sheath 18, this means being accessible at the proximal end of the tubes 22. This movement means is preferably connected to all of the tubes 22, so as to allow them to move simultaneously.

Each tube 22 can be radially deployed between a retracted configuration, in which each tube 22 can be covered by the sheath 18, and a deployed configuration radially protruding relative to the sheath 18.

Therefore, when the sheath 18 is in its first position, the tubes 22 remain in the retracted configuration, and their perforations 24 are covered by the sheath 18. However, when the sheath 18 is in its second position, the perforations 24 are exposed, and the tubes can go to their deployed configuration.

Advantageously, each tube 22 also has, near its distal end 22B, a cutting means 26, for example having a blade as shown in FIG. 4. This cutting means 26 can cut out a tumor when the tubes 22 radially deploy in said tumor, as will be described later.

It will be noted that at least one of the elements from amongst the prop 12, the tubes 22 or the sheath 18 is provided with graduations, indicating the movement of the tube 22 or the sheath 18 relative to the prop 12.

Preferably, graduations are formed on the prop 12, so as to measure the movement of the sheath 18 relative to said prop 12, and graduations are formed on the sheath 18, so as to measure the movement of the tubes 22 relative to said sheath 18.

Lastly, the medical device 10 has a means for injecting a medical substance intended to be connected to the proximal end 22A of each tube 22. For example, this injection means includes a vapor generator, connected to a pump or a syringe. When this injection means is activated, the medical substance is conveyed in the tubes 22 up to their perforations 24, from which this medical substance is injected into the tumor.

The operation of the medical device 10 will be described below.

First, the medical device 10 is inserted into the patient, through percutaneous access, until the nose 16 reaches a tumor 28 to be treated. The medical device 10 is manipulated using a handling member 14 and the prop 12.

The movement of the radiopaque nose 16 is observed by radiography, so as to guide the manipulation of the medical device 10.

During this insertion the sheath 18 remains in its first position, as shown in FIG. 1.

Once the tumor 28 is reached, the medical device 10 is maneuvered so that the nose 16 pierces this tumor and passes all the way through it.

The sheath 18 is then moved towards its second position, so as to expose the tubes 22, so that the pierced tumor is held between the sheath 18 and the nose 16, as shown in FIG. 2. It will be noted that the movement of the sheath 18 along the prop can be verified using graduations of the prop 12.

Once the sheath 18 is in its second position, the nose 16 is immobilized in position, via the prop 12, and the sheath 18 is then fixed in position relative to the prop 12, via an immobilizing means 20.

The tubes 22 are then pushed, owing to the means for moving these tubes 22, towards their distal ends 22B. Since the nose 16 and the sheath 18 are kept in position, the movement of the tubes 22 results in deploying them radially near their distal end 22B, as shown in FIG. 3.

The cutting means 26 of the tubes 22 then radially cuts out the tumor 28 during radial deployment of said means, which facilitates said deployment.

Once the tubes 22 are deployed optimally in the tumor 28, the tubes 22 then each extending in a C shape, the medical substance is injected into the tubes 22 using an injection means. This medical substance is then injected into the tumor 28 owing to the perforations 24, the diffusion of this substance being favored by the cuts made in the tumor. This injection then causes the reduction or destruction of the tumor 28.

It will be noted that the invention is not limited to the embodiment previously described, but could assume various alternatives without going beyond the scope of the claims.

1. A medical device (10), in particular to inject a fluid into a body (28), characterized in that it includes:
a prop (12) extending between a proximal end (12A) and a distal end (12B),
a nose (16) in the form of a perforating tip, secured to the distal end of the prop (12),
at least one injection tube (22) for injecting fluid into the body, the tube (22) extending between a proximal end (22A) and a distal end (22B), the tube (22) having at least one perforation (24) near its distal end (22B), and
a sheath (18), in which the prop (12) and the tube (22) are housed, the sheath (18) being able to be moved by sliding along the prop (12), between a first position in which the sheath (18) covers the perforation (24), and a second position in which the perforation (24) is exposed,
and in which:
each tube (22) is deployed radially between a retracted configuration, in which each tube (22) can be covered by the sheath (18), and a radially protruding deployed configuration, and
the proximal end (22A) of each tube (22) can be accessed outside the sheath (18), so that the deployment of each tube (22) towards its deployed position can be controlled from its proximal end (22A), the distal end (22B) of each tube being secured to the nose (16) or the prop (12).

2. The medical device (10) according to claim 1, having at least two fluid injection tubes (22), and means for jointly moving each tube (22) by sliding in the sheath (18), accessible at the proximal end (22A) of the tubes (22).

3. The medical device (10) according to claim 1, having a means (20) for immobilizing the sheath (18) in its second position relative to the prop (12).

4. The medical device (10) according to claim 1, wherein the sheath (18) is close to the nose (16) in its first position, preferably in contact with said nose (16), and the sheath (18) is spaced away from this nose (16) in the second position.

5. The medical device (10) according to claim 1, wherein each tube (22) has, near its distal end (22B), a cutting means (26), for example having a blade, able to cut out a tumor (28) when said tube (22) deploys radially in the tumor (28).

6. The medical device (10) according to claim 1, wherein at least one element from amongst the prop (12), the tube (22) or the sheath (18) is provided with graduations, indicating the movement of the tube (22) or sheath (18) relative to the prop (12).

7. The medical device (10) according to claim 1, wherein the nose (16) is made from a radiopaque material.

8. The medical device (10) according to claim 1, having a fluid injection means intended to be connected to the proximal end (22A) of each tube (22), for example comprising a vapor generator connected to a pump or a syringe.

9. The medical device (10) according to claim 2, having a means (20) for immobilizing the sheath (18) in its second position relative to the prop (12).

10. The medical device (10) according to claim 2, wherein the sheath (18) is close to the nose (16) in its first position, preferably in contact with said nose (16), and the sheath (18) is spaced away from this nose (16) in the second position.

11. The medical device (10) according to claim 3, wherein the sheath (18) is close to the nose (16) in its first position, preferably in contact with said nose (16), and the sheath (18) is spaced away from this nose (16) in the second position.

12. The medical device (10) according to claim 2, wherein each tube (22) has, near its distal end (22B), a cutting means (26), for example having a blade, able to cut out a tumor (28) when said tube (22) deploys radially in the tumor (28).

13. The medical device (10) according to claim 3, wherein each tube (22) has, near its distal end (22B), a cutting means (26), for example having a blade, able to cut out a tumor (28) when said tube (22) deploys radially in the tumor (28).

14. The medical device (10) according to claim 4, wherein each tube (22) has, near its distal end (22B), a cutting means (26), for example having a blade, able to cut out a tumor (28) when said tube (22) deploys radially in the tumor (28).

15. The medical device (10) according to claim 2, wherein at least one element from amongst the prop (12), the tube (22) or the sheath (18) is provided with graduations, indicating the movement of the tube (22) or sheath (18) relative to the prop (12).

16. The medical device (10) according to claim 3, wherein at least one element from amongst the prop (12), the tube (22) or the sheath (18) is provided with graduations, indicating the movement of the tube (22) or sheath (18) relative to the prop (12).

17. The medical device (10) according to claim 4, wherein at least one element from amongst the prop (12), the tube (22) or the sheath (18) is provided with graduations, indicating the movement of the tube (22) or sheath (18) relative to the prop (12).

18. The medical device (10) according to claim 5, wherein at least one element from amongst the prop (12), the tube (22) or the sheath (18) is provided with graduations, indicating the movement of the tube (22) or sheath (18) relative to the prop (12).

19. The medical device (10) according to claim 2, wherein the nose (16) is made from a radiopaque material.

20. The medical device (10) according to claim 3, wherein the nose (16) is made from a radiopaque material.
